(19) United States
(12) Patent Application Publication
(10) Pub. No.: US 2018/0267037 A1
(43) Pub. Date: Sep. 20, 2018

(54) METHOD FOR THE NONINVASIVE DETECTION OF ACTIVATED BIFIDOBACTERIA

(71) Applicant: EVOLVE BIOSYSTEMS INC., Davis, CA (US)

(72) Inventors: David KYLE, Gualala, CA (US); David MILLS, Davis, CA (US); Carlito LEBRILLA, Davis, CA (US)

(21) Appl. No.: 15/539,172

(22) PCT Filed: Dec. 23, 2015
(86) PCT No.: PCT/US15/00262

§ 371 Date: Jun. 23, 2017

Related U.S. Application Data
(60) Provisional application No. 62/096,117, filed on Dec. 23, 2014.

(51) Int. Cl. G01N 33/569; G01N 33/558; C07K 16/12

(52) U.S. Cl. CPC G01N 33/56911; G01N 33/558; G01N 33/60; G01N 2333/36; G01N 2469/20; C07K 16/1292

(57) ABSTRACT

Some embodiments of the invention include a kit and a device for detecting activated bifidobacteria. Methods of making and using the kit and/or device are also described herein.

### FIELD OF THE INVENTION

The embodiments described herein relate generally to healthcare, and more particularly, to a method for the noninvasive detection of activated bifidobacteria as a predictive tool of general health. For example, the embodiments may be related to the measurement of activated bifidobacteria in an infant fecal sample using an in-lab or point-of-care, noninvasive device and using that information to establish the presence or absence of a normal Milk Oriented Microbiota (MOM) (Mills, 2015, The FASEB Journal, 29(1 Supplement): p. 358-4).

### BACKGROUND

In Western countries, where there exists a high degree of medical care including excessive use of antibiotics and a high level of hygiene in the population, newborn infants are commonly found to be dysbiotic as defined herein. The dysbiosis of the infant intestinal microbiome leads to increased gastrointestinal damage and/or inflammation and/or delayed or altered immunological programming and tolerization and/or metabolic disruption locally or systemically. The consequences of early dysbiosis may have an impact throughout the entire life of that individual.

Certain bifidobacteria (preferably B. longum and more preferably B. longum subspecies infantis) can internalize and metabolize the complex oligosaccharides that make up about 15% of the energy content of human milk. These oligosaccharides, which are not processed by human gut enzymes, are referred to herein as human milk oligosaccharides (HMOs) and the consumption of the HMOs by these bifidobacteria leave little or no residual energy source available for the growth of other bacterial species in the lower colon of the breast-fed human infant. In the absence of these bifidobacteria species, a number of other species can grow uncontrollably leading to dysbiosis that can result in significant medical consequences. These bifidobacteria, specifically B. longum subsp. infantis are normally present at high concentrations (up to about 70% of the total microbiome) in the gut of a healthy, vaginally-delivered, breast-fed baby. The obstetric standard of care in a typical hospital today involves births from both Cesarean Section and vaginal delivery, followed by human milk or infant formula feeding for the baby. The cleanliness of the surgical suite and levels of hygiene for the mother pre- and post-op and for the newborn infant are such that it is unlikely that the infant will get seeded with the required bacteria normally found in the microbiome of the vagina/intestine, resulting in dysbiosis. Dysbiosis can alternatively be caused by infants losing the beneficial bifidobacterium as a result of illness or medical intervention (e.g., antibiotic treatment).

A major advantage of normal vaginal birthing and breast feeding is the nominal establishment of a protective Milk-Oriented Microbiota (MOM) that is dominated by specific strains of bifidobacteria, in particular B. longum subspecies infantis. Failure to initiate colonization due to C-section birth, antibiotic treatment at birth, or formula-feeding in the first 12 months of life can lead to a permanent shift in the microbiota away from the protective MOM in spite of breastfeeding. Furthermore, approximately 20% of all women are characterized as non-secretors of certain components of the HMOs in their milk and even when those women are actively nursing their infant, there still may be a shift in the microbiota away from the protective MOM.

Detecting B. longum subspecies infantis within infants or their fecal samples is critical to evaluating the microbiota status of infants at risk for various pathogenic, immunological, and metabolic disorders; however, there is no rapid and accurate means of detecting specific bacterial species in a complex mixture of microorganisms, such as those found in feces, that can be monitored in real time at the point-of-care or in a lab to allow for appropriate and timely intervention. Culturing methods are too slow and require samples to be sent away for analysis, and typically fail to detect delicate organisms including Bifidobacteria. Sequencing and molecular methods are expensive and require skilled technicians and complex data analysis to monitor the infant microbiota. Additionally, there is currently no point-of-care method to document the recolonization and establishment of a MOM-dominated infant microbiota when using probiotic, prebiotic, and symbiotic interventions designed to restore a breast fed appropriate microbiota in infants.

Therefore, what is needed is a diagnostic platform designed to detect the presence of activated B. longum subspecies infantis in a simple and noninvasive fashion using s a sample of the infant's feces to determine the microbiome of the lower bowel of the infant, and to define the extent of the infant's MOM.

### SUMMARY

This invention provides a kit suitable for use in detecting activated bifidobacteria. The kit comprises an antibody and/or aptamer that specifically binds an antigen and/or biopolymer associated with, found on, or substantially identical to that found on, activated bifidobacteria. The antigen and/or biopolymer can be an antigen and/or biopolymer found on activated bifidobacteria that is not present in a nonactivated bifidobacteria, and, optionally, does not bind any antigen found on non-activated bifidobacteria. In some embodiments, the antibody can be a monoclonal and/or a polyclonal antibody.

The antigen found on activated bifidobacteria can be a protein. The antigen may be from bifidobacteria that was activated by its growth in the presence of mammalian milk oligosaccharides. The antigen can comprise a protein encoded by at least one gene selected from, or substantially identical to, the group consisting of Blon0015, Blon0883, Blon2344, Blon2347, and Blon2350. The antigen and/or biopolymer can be from bifidobacteria that was activated by its growth in the presence of mammalian milk oligosaccharides. The bifidobacteria can be B. longum, for example, B. longum subspecies infantis. The protein may be part of the bacterium, a fragment, or isolated from the bacterium.

The kit may also include a test strip, which may include a blotting pad, a conjugate pad, a test band, a control band, and/or a soak pad. The test strip can be a dipstick. If the kit comprises a test strip, at least a portion of the antibody can be covalently bound to the test strip.

The antibody, or a portion of the antibody, in the kit may comprise a detectable marker such as, but not limited to, a visually detectible marker, a fluorescent marker, a magnetic marker, and/or a radioactive marker. The kit may also include a mechanism for collecting the antigen and/or biopolymer associated with or found on activated bifidobacteria. The mechanism for collecting the antigen and/or biopolymer can collect the antigen and/or biopolymer from fecal matter, for example, human fecal matter.

The invention provides a device comprising an antigen and/or biopolymer associated with or found on, or substantially identical to that found on or associated with, activated bifidobacteria. The antigen can be an antigen and/or biopolymer associated with or found on activated bifidobacteria that is not present in a nonactivated bifidobacteria. The device also comprises an antibody and/or aptamer that specifically binds the antigen, and, optionally, does not bind any antigen and/or aptamer found on non-activated bifidobacteria.

In some embodiments, the device includes a detectable marker, which may be associated with an antigen and/or biopolymer or with an antibody and/or aptamer, such as a visually detectible marker, a fluorescent marker, and/or a radioactive marker. In some embodiments, the antigen and/or biopolymer is from bifidobacteria that was activated by its growth in the presence of mammalian milk oligosaccharides such as but not limited to bovine milk oligosaccharides or human milk oligosaccharides. In some embodiments of the device, the antigen and/or biopolymer is directly bound to the antibody and/or aptamer.

The antigen and/or biopolymer in the device can be an intact protein, a fragment thereof, or any modification thereof. The antigen and/or biopolymer can include a protein or a fragment thereof encoded, or substantially equivalent to a protein encoded, by at least one gene selected from the group consisting of Blon0015, Blon0883, Blon2344, Blon2347, and Blon2350. The antigen and/or biopolymer can be from bifidobacteria that was activated by its growth in the presence of mammalian milk oligosaccharides. The bifidobacteria can be B. longum, for example, B. longum subspecies infantis. In some embodiments, the antibody can be a monoclonal and/or a polyclonal antibody.

The device may comprise a test strip, which can include at least one of a blotting pad, a conjugate pad, a test band, a control band, and a soak pad. The test strip can be a dipstick. If the kit comprises a test strip, the antibody and/or aptamer can be covalently bound to the test strip.

Any of the kits or devices described herein can be made by producing the antibody and/or aptamer to the antigen and/or biopolymer and placing the antibody and/or aptamer on the test strip. At least a portion of the antibody and/or aptamer can be covalently bound to the test strip.

Any of the kits described herein can be used to detect the presence of activated bifidobacteria by collecting a fecal sample and applying the fecal sample or at least a protein-containing portion of the fecal sample to the test strip. The fecal sample can be a human fecal sample, for example, from a human infant less than 18 months post-conceptual age.

### DETAILED DESCRIPTION OF THE FIGURES

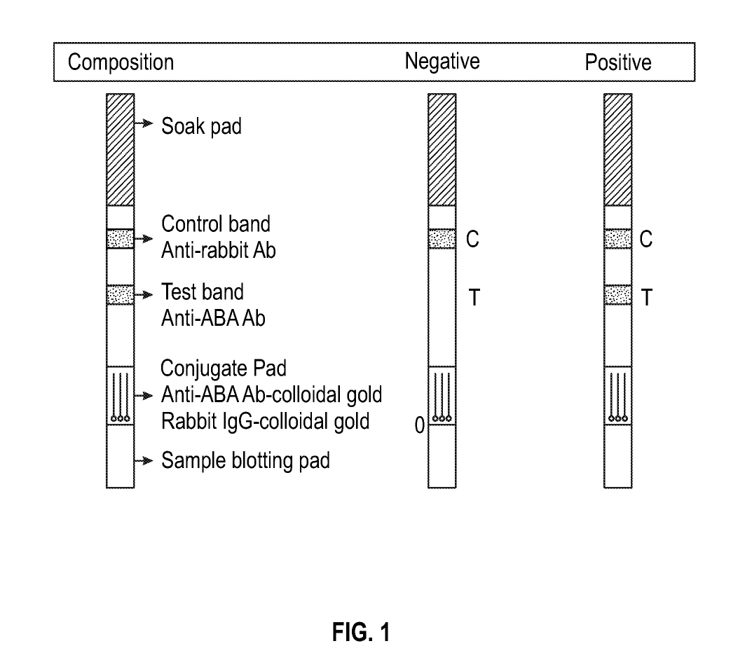

FIG. 1 shows an embodiment of a kit and/or device of the present invention.

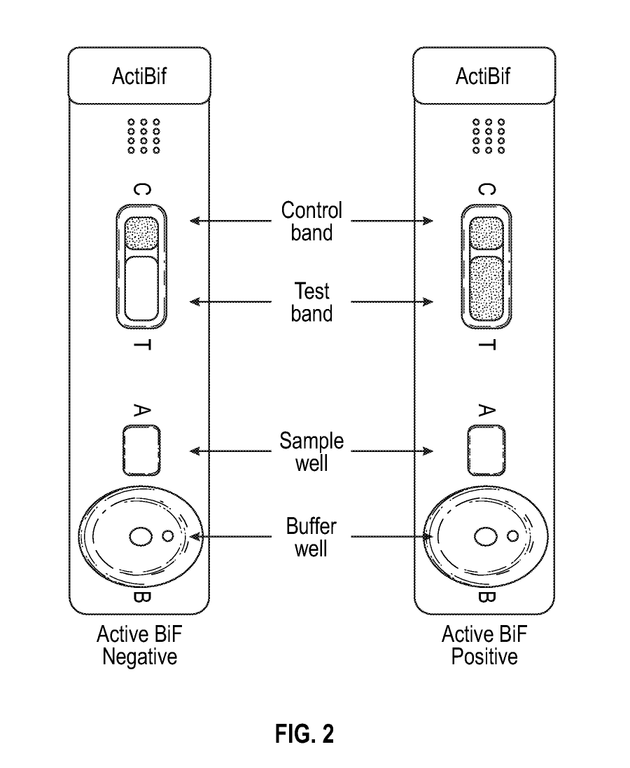

FIG. 2 shows an embodiment of a kit and/or device of the present invention.

### DETAILED DESCRIPTION

In the following detailed description of the invention, numerous details, examples, and embodiments of the invention are described; however, it will be clear and apparent to one skilled in the art that the invention is not limited to the embodiments set forth and that the invention can be adapted for any of several applications.

The phrase “activated bifidobacteria” is defined herein as the state of the cells, as measured by the up-regulation or down-regulation of genes and their coded polypeptides including but not limited to those coding for oligosaccharide binding proteins, transport proteins, and enzymes responsible for the degradation of the complex milk oligosaccharides, which provides significant benefits to a human, such as a newborn infant. Such beneficial characteristics of the activated bifidobacteria include, but are not limited to, one or more of a higher binding affinity of the bacteria to the gut mucosa, higher colonization of the gastrointestinal tract thereby preventing any significant growth of other bacterial clades, the ability to consume complex oligosaccharides, and/or a greater stimulation of the immune response as measured by positive alterations of immune response markers, relative to the organism in a pre-activated state (Lewis, et al., 2015, Microbiome, 3:13; Huda, et al., 2014, Pediatrics, 134:2 e362-e372). For additional information regarding activated bifidobacteria, see PCT/US2015/057226, filed Oct. 23, 2015, the disclosure of which is hereby incorporated in its entirety.

The phrase “dysbiosis” is defined herein as the state of microbiomal deficiency inside the body, resulting from an insufficient level of keystone bacteria (e.g., bifidobacteria, such as B. longum subsp. infantis) or can overabundance of harmful bacteria in the gut. For example, an insufficient level of keystone bacteria (e.g., bifidobacteria, such as B. longum subsp. infantis) may be a level at which a person lacks suitable colonization of the bifidobacteria in the gut (for example, around 10⁶ cfu/g stool or less). Dysbiosis in a human infant is defined herein as a microbiome that does not comprise B. longum subsp. infantis at levels of at least 10⁸ cfu/g fecal material during the first 12 months of life.

The invention provides kits or devices capable of determining the absolute or relative abundance of specific strains of bifidobacteria naturally found in the breast fed infant intestine using a detection process capable of binding to a unique property of the bacteria and transducing a signal (colorimetric, voltaic, auditory) resulting from the detection of that unique property typically comprising a gene product (e.g., an antigen) found on, or substantially identical to that found on, activated bifidobacteria.

This invention contemplates kits for the measurement or identification of the presence of various strains of bifidobacterial species, including B. bifidum, B. longum, B. breve, B. pseudocatenultaum, and B. longum subspecies infantis, hereinafter collectively referred to as Milk Oriented Microbiota (MOM), and related microorganisms in the feces of infants or other individuals. In one or more embodiments, the devices and methods of this invention will identify the presence of activated bifidobacteria, where activation is defined by a specific response of the MOM to complex dietary glycans (DGs), which may be present in mammalian milk, including human milk and bovine milk. The activation response may be the up-regulation of certain genes responsible for the production of proteins (e.g., binding proteins or hydrolytic enzymes) responsible for the uptake and metabolism of the DGs by the bifidobacteria and for the metabolites produced as a result of the metabolism of specific glycans and proteins. Metrics for the identification of the presence of activated Bifidobacteria longum subspecies infantis and related bacteria include: the expression of specific gene products, or fractions thereof, which are expressed in the activated state, including glycosidases, solute binding proteins, oligosaccharide degradation products, ribonucleic acid (RNA), and solute transporters. The invention may also be used to measure and monitor the absence of keystone bacteria (i.e., species that play central roles in the functioning of ecosystems, such as B. longum subspecies infantis in infant nutrition) normally expected in the vaginally-delivered and breast-fed infant and the success of interventions designed to re-colonize and establish a protective MOM found in the vaginally delivered and breast fed infants.

The antibodies for use herein may include polyclonal antibodies, monoclonal antibodies, antibody fragments, or combinations thereof. For the purposes of this invention, antibodies may be substituted by aptamers which bind to antigens of activated bifidobacteria with comparable levels of affinity and specificity. See Mayer, G., “The Chemical Biology of Aptamers,” Angew. Chem. Int. Ed. Engl., 2009, 48(15):2672-2689. The antibodies and/or aptamers for use herein may be labeled with a marker, which may be, or may generate, a visually, acoustically, electronically or magnetically detectable signal. For example, the marker can be a fluorescent marker, a radioactive marker, or combinations thereof.

Where the specific gene product expressed by bifidobacteria in the activated state is a ribonucleic acid, such as a mRNA, techniques for determining the presence and amount of particular sequences (such as microarrays) are well known to the skilled person. Quantitative assays from nucleic acids are known in the art, and the skilled person can readily quantitate bifidobacteria by applying one of those methods to the known sequence of one or more bifidobacterial genes. See, e.g, Sela et al., “The Genomic Sequence of Bifidobacterium longum subsp. infantis Reveals Adaptations for Milk Utilization within the Infant Microbiome,” 2008, Proc. Nat. Acad. Sci. U.S.A., 105(48):18964-18969.

The device of the present invention may be a noninvasive device, such as a Point-of-Care (POC) test kit, such as a standard dipstick, lateral flow device, flow through device, or as laboratory tests including microfluidics based analyte assays (“lab-on-a-chip”), qPCR, and enzyme tests (e.g., fructose-6-phosphate phosphoketolase).

For example, a dipstick, lateral flow or flow through device may be tested by being introduced to, for example, from about 1 ul to about 1000 ul of a stool sample, preferably 10-500 uL, more preferably 50-200 uL of a stool sample, and may demonstrate specific serotype specific detection with from about 75 to about 100%, preferably about 85 to about 100%, more preferably about 95 to about 100%, specificity and sensitivity.

The device and method of the present invention may be used for a number of different purposes, including: (i) measuring the presence and relative abundance of activated Bifidobacterium longum subspecies infantis in samples from infants, children, or adult patients under treatment with probiotics; (ii) detecting the presence and functions of activated bifidobacteria together with milk derived oligosaccharides when intended to be used in order to restore the normal population of bacterial microbiota within the intestine; (iii) measuring activated bifidobacterium species as commensal bacteria levels in the intestine of infants recovering from the depletion in the microbiota caused by antibiotic treatment for infection, sepsis, or by any other pathological, metabolic, or genetic condition; (iv) measuring activated bifidobacteria with or without oligosaccharides that are used for treating infants and patients undergoing antibiotic treatment; (v) measuring activated bifidobacteria by measuring the abundance of microbe associated enzymes, such as glycosidases, solute binding proteins, and solute transporters that are diagnostic of the intestinal functions of bifidobacteria growing on indigestible disaccharides and oligosaccharides; (vi) measuring activated bifidobacteria status prior to interventions associates with successful immune responses including vaccinations, introduction of complementary foods, and allergens; (vii) measuring activated bifidobacteria as a diagnostic metric to prevent or treat necrotizing enterocolitis (NEC) and intestine originated sepsis subsequent to and/or immediately following the cessation of antibiotic treatment for a separate infection; (viii) measuring activated bifidobacteria supplement efficacy when intended to be used either as supplements, formula, or food additives in order to enrich infant's intestinal microbiota; (ix) measuring activated bifidobacteria in infants fed with infant formula; (x) measuring activated bifidobacteria in clinical settings to monitor interventions designed to restore the microbiota of infants and adults treated for intestinal infections, such as colitis and Crohn's disease also associated with unbalanced bacterial population throughout the intestine; and/or (xi) measuring activated bifidobacterial status in pre- and post-surgery infants, children, and adults in order to assess the risk of sepsis and/or the recommendation for appropriate supplementation. The device may also be used for the identification of MOMs in the feces of other animals including, mammals, such as companion animals, performance animals, and agriculturally important animals.

Preparing the composition comprising a protein antigen and/or biopolymer from an activated bifidobacteria, which is optionally not present in a nonactivated bifidobacteria, and an antibody and/or aptamer to that antigen and/or biopolymer, where the antigen and/or biopolymer may be non-covalently bound to the antibody and/or aptamer, may comprise: (i) selecting a strain of bifidobacteria by combining a human infant fecal sample with human milk oligosaccharides and isolating fast growing colonies; (ii) identifying genes that are upregulated in the bifidobacteria when cultivated in a medium containing human milk oligosaccharides as the primary carbon source; (iii) isolating and purifying the products of those up-regulated genes, (iv) preparing at least one antibody and/or aptamer (which may be a monoclonal and/or polyclonal antibody), to the protein products of the up-regulated genes; (v) labeling the at least one antibody and/or aptamer with a visually, acoustically, or electronically detectable marker; and/or (vi) combining the at least one monoclonal and/or polyclonal antibody with the protein antigen.

A human (e.g., a human infant) that lacks suitable colonization of the bifidobacteria in the gut may still have a level of bifidobacteria of around 10⁶ cfu/g stool. Colonization of the human (e.g., a human infant) with bifidobacteria (e.g., B. longum subsp. infantis) by supplementing said human with DGs that are exclusively consumed by said bifidobacteria, will result in 10⁸ cfu/g stool or greater levels of bifidobacteria in the gut. Colonization by activated bifidobacteria produces a level of bifidobacteria in the stool of at least 10⁸ cfu/g stool, more likely at least 10⁹ cfu/g stool, and typically 10¹⁰ cfu/g stool or greater. Thus, an individual with levels of bifidobacteria that are greater than 10⁸ cfu/g stool, for example, at least 10⁹ cfu/g stool, or at least 10¹⁰ cfu/g stool has activated bifidobacteria.

Some embodiments of the present invention comprise a method and device to identify and distinguish those infants who have acquired a complete and protective MOM that is normally associated with breast feeding compared to those who have not acquired the protective MOM, the device and method comprising using various analytical platforms including sequencing, mRNA expression, specific protein analysis by antibody binding, and metabolic determination. The analytical platforms may all be employed to detect sequences, mRNA's for specific proteins, specific proteins, and metabolites that are quantitatively related to the presence and abundance of activated MOM, such as bifidobacteria, particularly B. longum subspecies infantis and activated B. longum subspecies infantis, in the feces of an infant or other individual.

The diagnostic platform of the present invention may be used to determine the status of the infant's microbiota during the first months of life, particularly after feeding of formula or following the administration of antibiotics or viable bacteria as intervention. The results of the measurement (i.e., the presence or absence of the MOM in the infant's feces) may be used to: (i) identify infants after birth that are not developing their appropriate MOM correctly; (ii) identify infants who have been fed transiently with formula and have lost their MOM; (iii) identify infants who have been treated with antibiotics and have lost their MOM due to the antibiotic; (iv) identify mothers (non-secretors) who may not be secreting sufficient DGs to establish a thriving community of MOMs in their infants; and (v) follow the microbiota of infants who have been found in cases (i)-(iv) above to be lacking appropriate MOM composition and have been treated with various live bacteria as intervention to correct this dysbiosis.

Some embodiments of the invention may also provide a mechanism to ensure a normal MOM in infants, children, and adults who are prescribed antibiotics for infection and in whom all normal commensal bacteria may be expected to be severely reduced or eliminated as a side effect caused by the desired efficacy of the antibiotic toward pathogens. The bacteria may also have acquired genetic elements that confer resistance to the antibiotics typically used in hospital and clinical practice.

Suitable specific sequences, genes, and proteins that may be a diagnostic indicator for the presence of B. longum subspecies infantis include those that may be used as markers to predict an activated state for the microbe. Suitable biomarkers include up-regulated genes coding for proteins, such as galactosidases, fucosidases, sialidases, hexose aminidases, permeases, and extracellular solute binding proteins (SBPs). Such genes may include, but are not limited to, Blon 35, Blon 653, Blon 881, Blon 2183, Blon 2340, Blon 2343, Blon 2475, Blon0042 (regulatory protein), BlonR0025 (tRNA), BlonR0017 (tRNA), BlonR0021 (tRNA), BlonR0022 (tRNA), Blon0083 (SBP), Blon2350 (SBP), Blon2347 (SBP), Blon2354 (SBP), Blon2344 (SBP), Blon2352 (SBP), Blon2351 (SBP),), and genes encoding proteins with the following protein family (Pfam) motifs including: Pfam00449 (urease alpha), Pfam1120 (fucosidase) Pfam0157 (SBP fam 1), and Pfam02012 (sialidase).

This list of constituent elements is intended to be exemplary only, and it is not intended that this list be used to limit the method of the present application to just these elements. Persons having ordinary skill in the art relevant to the present disclosure may understand there to be equivalent elements that may be substituted within the present disclosure without changing the overall invention.

### EXAMPLE

The various elements of the method of the present invention may be related in the following exemplary fashion. It is not intended to limit the scope or nature of the relationships between the various elements and the following examples are presented as illustrative examples only.

A Lateral Flow Assay (LFA) for the detection of the presence of activated B. longum subspecies infantis and based on the principle of immune-chromatography is shown and described in FIG. 1. An antibody is prepared in rabbits to the selected antigen(s) whose expression is induced by the activation of the B. longum subspecies infantis during its cultivation in a medium comprising bovine milk oligosaccharides (BMOs). Such an antigen could be an induced regulatory protein (e.g., the product of Blon0015) or more preferably a cell surface solute binding protein (e.g., the product of Blon2347). This anti-ActiveBifAntigen antibody (anti-ABA-Ab) is then labeled with colloidal gold using methods known in the art.

A test sample of fecal material is mixed with a lysing buffer or clearing buffer and applied to the sample blotting pad, which also acts to filter small particles that may be carried along the membrane. The clarified sample flows through the membrane assembly of the device to the conjugate pad comprising a colored ABA-Ab-colloidal gold conjugate, which then complexes with the ABA from the lysed fecal sample. This complex then moves further on the membrane to the test region where it is immobilized by an unlabeled anti-ABA-Ab, which has been fixed on the membrane in the test region thereby forming a sandwich in the test region and leading to the formation of a pink-colored band, which confirms a positive test. Absence of this colored band in the test region indicates a negative test result. Unreacted conjugate and unbound complex, if any, moves further up on the membrane to the Control band. Anti-rabbit antibodies fixed on the membrane in the control band react with the rabbit IgG traveling along with the unreacted/unbound complex, forming a second colored band. This control band serves to validate the test performance. Any remaining material continues to move further on the membrane and is subsequently absorbed at the soak pad.

The above process may also be incorporated into a single disposable and non-reusable unit, such as those used for pregnancy testing or as described in FIG. 2.

The above-described embodiments and examples of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims. Any reference specifically identified herein is incorporated by reference, at least to the extent needed to fully enable the invention.

What is claimed is:

1. A kit comprising an antibody, wherein the antibody specifically binds an antigen associated with or found on activated bifidobacteria.

2. The kit of claim 1, wherein the antigen associated with or found on bifidobacteria does not bind any antigen found on non-activated bifidobacteria.

3. The kit of any one of claim 1 or 2, further comprising a test strip.

4. The kit of claim 3, wherein the test strip comprises at least one of a blotting pad, a conjugate pad, a test band, a control band, and a soak pad.

5. The kit of any one of claim 3 or 4, wherein the test strip is a dipstick.

6. The kit of any one of claims 1-5, wherein the antibody comprises a detectable marker.

7. The kit of claim 6, wherein the detectable marker is a visually detectable marker.

8. The kit of any one of claim 6 or 7, wherein the detectable marker is fluorescent.

9. The kit of any one of claims 6-8, wherein the detectable marker is radioactive.

10. The kit of any one of claims 1-9, wherein the antibody is a monoclonal antibody.

11. The kit of any one of claims 1-10, further comprising a mechanism for collecting the antigen and/or biopolymer associated with or found on activated bifidobacteria.

12. The kit of claim 11, wherein the mechanism for collecting the antigen collects the antigen from fecal matter.

13. The kit of claim 12, wherein the fecal matter is human fecal matter.

14. The kit of any one of claims 1-13, wherein the antigen is a protein.

15. The kit of claim 1-13, wherein the antigen comprises protein encoded by at least one gene selected from the group consisting of Blon0015, Blon0883, Blon2344, Blon2347, and Blon2350.

16. The kit of any one of claims 1-15, wherein the antigen is from bifidobacteria that was activated by its growth in the presence of mammalian milk oligosaccharides.

17. The kit of any one of claims 1-16, wherein the bifidobacteria is B. longum.

18. The kit of any one of claims 17, wherein the bifidobacteria is B. longum subspecies infantis.

19. The kit of any one of claims 3-18, wherein the antibody is covalently bound to the test strip.

20. The kit of any one of claims 1-19, wherein the antigen is detected in an amount indicating bifidobacteria at a level of at least 10⁸ cfu/g stool, preferably at least 10⁹ cfu/g stool, more preferably at least 10¹⁰ cfu/g stool.

21. A device comprising an antigen and/or biopolymer associated with or found on activated bifidobacteria and an antibody and/or aptamer that specifically binds the antigen and/or biopolymer.

22. The device of claim 21, wherein the antibody and/or aptamer does not bind any antigen and/or biopolymer found on non-activated bifidobacteria.

23. The device of any one of claim 21 or 22, wherein the antigen and/or biopolymer is bound to the antibody and/or aptamer.

24. The device of any one of claims 21-23, wherein the antigen and/or biopolymer is a protein.

25. The device of claim 24, wherein the antigen and/or biopolymer comprises protein encoded by at least one gene selected from the group consisting of Blon0015, Blon0883, Blon2344, Blon2347, and Blon2350.

26. The device of any one of claims 21-25, wherein the antigen and/or biopolymer is from bifidobacteria that was activated by its growth in the presence of mammalian milk oligosaccharides.

27. The device of claim 21-26, wherein the bifidobacteria is B. longum.

28. The device of any one of claims 27, wherein the bifidobacteria is B. longum subspecies infantis.

29. The device of any one of claims 21-28, wherein the antibody is a monoclonal antibody.

30. The device of any one of claims 21-29, wherein the antibody and/or aptamer comprises a detectable marker.

31. The device of claim 30, wherein the marker is a visually detectable marker.

32. The device of any one of claim 30 or 31, wherein the detectable marker is fluorescent.

33. The device of any one of claims 30-32, wherein the detectable marker is radioactive.

34. The device of any one of claims 21-33, further comprising a test strip.

35. The device of claim 34, wherein the test strip comprises at least one of a blotting pad, a conjugate pad, a test band, a control band, and a soak pad.

36. The device of any one of claim 34 or 35, wherein the test strip is a dipstick.

37. The device of any one of claims 21-36, wherein the antigen and/or biopolymer is detected in an amount indicating of bifidobacteria at a level of at least 10⁸ cfu/g stool, preferably at least 10⁹ cfu/g stool, more preferably at least 10¹⁰ cfu/g stool.

38. A method of making the kit of any of claims 3-20, wherein the method comprises producing the antibody to said antigen and placing the antibody on the test strip.

39. The method of claim 38, wherein at least a portion of the antibody is covalently bound to the test strip.

40. A method of using the kit of any one of claims 3-20, comprising collecting a fecal sample and applying at least a protein-containing portion of said fecal sample to the test strip.

41. The method of claim 40, wherein the fecal sample is a human fecal sample.

42. The method of claim 41, wherein said human is a human infant less than 18 months post-conceptual age.

43. A method of preparing an apparatus comprising an activated protein antigen and an antibody to that antigen wherein the antigen is non covalently bound to the antibody, by:
a. selecting a strain of bifidobacteria by combining a human infant fecal sample with human milk oligosaccharides and isolating fast growing colonies;
b. identifying genes that are up-regulated in the bifidobacteria when cultivated in a medium containing human milk oligosaccharides as the sole carbon source;
c. isolating and purifying the products of those up-regulated genes;
d. preparing a monoclonal antibody to the protein products of the up-regulated genes;
e. labeling the monoclonal antibody with a visually, acoustically or electronically detectable marker; and
f. combining the monoclonal antibody with the protein antigen.
